(19) United States
(12) Patent Application Publication
(10) Pub. No.: US 2023/0241422 A1
(43) Pub. Date: Aug. 3, 2023

(54) METHODS TO TREAT CANCER, INFLAMMATIONS AND TUMORS

(71) Applicant: John F. Warlick, Kennesaw, GA (US)

(72) Inventor: John F. Warlick, Kennesaw, GA (US)

(21) Appl. No.: 18/123,003

(22) Filed: Mar. 17, 2023

Related U.S. Application Data
(63) Continuation-in-part of application No. 17/097,166, filed on Nov. 13, 2020.
Application No. 17/097,166 is a continuation-in-part of application No. 16/879,979, filed on May 21, 2020.
(60) Provisional application No. 62/852,683, filed on May 24, 2019.

(51) Int. Cl. A61N 7/00

(52) U.S. Cl. CPC A61N 7/00; A61N 2007/0004

(57) ABSTRACT

A method of treating a patient having cancer cells has the step of directing sound energy from one or more acoustic shock wave or pressure pulse treatments into the patient targeting the cancer cells in at least one tumor or in the blood to cause the patient’s immune system to identify the cancer cells as foreign objects and trigger a natural healing mechanism of the patient’s immune system to destroy the foreign objects. Wherein targeting the at least one tumor initiates cellular communication resulting in an unmasking of all cancer cells in the patient’s body by destroying a cloaking capability of the cancer cells which made the cancer cells indistinguishable from normal cells allowing the cancer cells to hide from the immune system prior to the one or more acoustic shock wave or pressure pulse treatments.

### RELATED APPLICATIONS

The present invention is a continuation in part of 17/097,166 filed on Nov. 13, 2020; which is a continuation in part of U.S. application 16/879,979 filed on May 21, 2020 entitled, “Device And Methods To Destroy Bacteria, Molds, Fungi And Viruses And For Reducing Inflammation And Markers In Organs And Tissue And To Extend The Utility Of Antibiotics” which claims priority to provisional application US 62/852,683 filed on May 24, 2019.

### FIELD OF THE INVENTION

The disclosure relates to the use of sound waves, more particularly acoustic shock waves or pressure pulses to destroy cancer cells and for reducing inflammation and markers in organs and tissue and to extend the utility of medications.

### BACKGROUND OF THE INVENTION

Cancer cells have the ability to hide in plain sight in the body. For many years, scientists have been working on ways to get cancer cells to come out of hiding in order to target them for destruction.

Cancer cells camouflage themselves by preventing the immune system from detecting cancer cells that are not supposed to be there. Our body is equipped with a powerful defense mechanism made up of immune cells. A type of immune cell, T-cells, act like warriors, detecting and destroying any suspicious-looking foreign cells. When a bacteria or virus enters the body, the substances released by T-cells, called antigens, are picked up by another type of immune cell, known as antigen-presenting cells (APCs). These APCs display the foreign antigens on their surface to show T-cells that a malicious cell was detected. T-cells have receptors on their surface that can engage with these antigens on foreign cells and kill them. Cancer cells are not foreign cells, but are our own body cells that have gone rogue due to various undesired mutations.

T-cells undergo a process of maturation, when they are strictly trained to differentiate cells as self and non-self in order to prevent them from harming normal body cells. Immune cells recognize tumors by detecting molecules present on the surface of cancer cells, known as neoantigens. Neoantigens are ‘faulty’ molecules that are made as the result of changes to the genetic code (mutations) within cancer cells.When immune cells scan the body, they recognize these faulty molecules as being non-self, which triggers an immune response against the cancer. When the receptor on a T-cell detects a self cell, it ignores the cell and gets deactivated. The normal body cells produce a protein known as PD-L1 (programmed death ligand 1), that identifies and prevents them from being destroyed by T-cells. Cancer cells have learned to use this to their advantage by producing PD-L1 in excess on their surface. When the receptor PD-1 on a T-cell detects the presence of PD-L1 on a cancer cell during immune surveillance, it sees the cancer cell as a normal cell and it is left unharmed in a process called immune checkpoint.

Scientists have been working for decades to make cancer cells come out of hiding and make them visible to immune cells for destruction. It was discovered that different immune checkpoint mechanisms can be targeted to eliminate cancer cells. These drugs reverse a cloaking mechanism that many types of cancer cells use to avoid being discovered by the immune system.

Cancer immunotherapy involves using immune checkpoint inhibitors’ to suspend the process that stops T-cells from killing cancer cells. One example is the drug Durvalumab, a monoclonal antibody that can specifically bind to the immune checkpoint molecule PD-L1. Binding of PD-L1 prevents its interaction with T-cells, which blocks the immune checkpoint and allows T-cells to kill the cancer cells containing PD-L1. Durvalumab has been approved for immunotherapy by the FDA (US Food and Drug Administration) for urothelial bladder cancer and has shown promise in early-stage studies for the treatment of non-small cell lung cancer and head and neck cancers. Cancer immunotherapy has been gaining popularity due to its great success in advanced melanoma, or cancer of the melanin pigment-producing cells; and its innovative mechanism of action, which uses the immune system to target cancer cells.

Research has shown that not all cancers are susceptible to immunotherapy. The success rates for some cancers, such as colon cancer and breast cancer, are very low. Another potential concern with using immunotherapy is the side effects that may be caused by it. Since immunotherapy fundamentally makes cancer cells more susceptible to attack by immune cells, it poses a risk of revving up the immune system. Thus, immunotherapy could lead to an increase in inflammation and cause itching, flu-like symptoms, and fatigue.

In other cases, cancer cells genetically change and evolve over time. Researchers discovered that as they evolve, they may lose the ability to create a protein known as interleukein-33, or IL-33. When IL-33 disappears in the tumor, the body’s immune system has no way of recognizing the cancer cells and they can begin to spread, or metastasize.

Cancer cells also have been found to use fibrin, an insoluble protein that creates a mesh that stops blood flow, as a protective coating that prevents the immune system from getting to the cell or tumor to destroy the cancer cells.

Almost all living creatures including plants are formed of cellular tissues. In virtually every living being these cellular communities form an outer protective barrier of tissues. In mammals this protective barrier is commonly referred to as skin or membrane. Similarly, in vegetables and plants the outer shell is really a protective barrier of skin or a peel that grows as the vegetable or fruit matures providing a shield from intrusions to the underlying and generally more vulnerable inner tissue. For example, in citrus fruits the juicy high liquid content of these tissues would be impossible to mature without the protective outer peel. Accordingly, the use of such natural shields or barriers to protect more vulnerable cells or tissue is the norm.

It is therefore of little surprise that on the molecular level cells whether aerobic or anaerobic have generally been known to exhibit an outer protective cellular membrane similar to a skin and any treatment to destroy such a cell typically required identifying certain cell membrane structures and targeting or weakening or penetrating this outer membrane. Once penetration occurred the viability of the organism was diminished resulting in a cessation of viability. This is particular true of cancer cells.

Cancer cells have a very strong and evolutionary desire to survive and thus is one of the more adaptive organisms found on earth. Mutant strains of cancer cells are commonly feared because of their huge capacity to adapt to threats particularly those involving the use of microbial disinfectants and antibiotics used to fight disease. One major concern in eradicating cancerous tumors or cells is the fact that these cells, when attacked by a surgical procedure or any treatment that ruptures the cell membranes can cause the cancer cells to activate a survival response causing rapid cell replication and cancer cell growth making the cancer spread throughout the body. For these reasons, most treatments require radiation and chemotherapy before and after surgical procedures. Recently, some have proposed the use of acoustic shock waves to destroy tumors, however, those require destroying the cancer cell membranes creating fragments by microbubbles created by high cavitation effects. The concern is this type of surgical procedure may cause a spreading of the disease.

Cancer cells grow through a form of cellular division. Blood agar cultures are used to grow colonies of cancer cells. The cluster starts out invisible to the naked eye and within 24 to 48 hours can be a large colony of millions of such cells. This has always been a well known phenomenon and concern.

Almost all of the prior art literature on the subject of eliminating or preventing cancer cells suggests one or more drugs or chemical agents as the solution to this problem.

What is sorely lacking are safe and reliable devices and methods to break down the cellular barrier properties of these cells by unmasking the cells allowing the immune system to identify and kill the cells while avoiding the risk of spreading the disease.

It is a further objective to enhance the use of medications to better attack and destroy the cancer cells without losing effectiveness due to mutations of the disease becoming resistant to the medications like antibiotics, chemotherapy or radiation.

### SUMMARY OF THE INVENTION

A method of treating a patient having cancer cells has the step of directing sound energy from one or more acoustic shock wave or pressure pulse treatments into the patient targeting the cancer cells in at least one tumor to cause the patient’s immune system to identify the cancer cells as foreign objects and trigger a natural healing mechanism of the patient’s immune system to destroy the foreign objects. Wherein targeting the at least one tumor initiates cellular communication resulting in an unmasking of all cancer cells in the patient’s body by destroying a cloaking capability of the cancer cells which made the cancer cells indistinguishable from normal cells allowing the cancer cells to hide from the immune system prior to the one or more acoustic shock wave or pressure pulse treatments. Treating the at least one tumor causes the cancer cells to secrete exosomes into extracellular spaces with MRNA that triggers a biologic cascade and cellular communication or signaling alerting the immune system to identify the cancer cells as foreign objects to target and destroy the cancer cells. The cellular communication or signaling is a secretion by the cancer cells to send exosomes outside of the cancer cells with proteins that the unmasking the cloaking capability of the cancer cells initiates causing the biologic cascade.

During the acoustic shock wave or pressure pulse treatment no cancer cells are destroyed by the sound energy, however soon thereafter the unmasking the cancer cells in the at least one tumor allows the immune system to attack all cancer cells.

The treatment includes an improved long and short term blood supply, these mechanisms include both short and long term improvements in blood supply, an up regulation of anti-microbial peptides, especially peptide LL 37, a disruption of biofilms that protect these foreign objects, and an increase in cellular communication such that healthy cells identify these foreign objects as targets of the body’s natural defenses. This improved blood supply allows a body to deliver natural defenses and increases the supply of medications administered by a physician or other medications, a disruption of cellular membranes, increased cell membrane permeability, and an improvement in cellular communication causing the patient’s immune system to identify and attack the cancer cells and further causes recruiting or stimulating an increase in anti-microbial peptides.

In one embodiment, the method further has the step of treating a reflexology zone to reduce systemic inflammation to accelerate cancer recovery.

The method further may include the step of administering one or more of medications, chemotherapy, radiation or checkpoint inhibitors to the patient including, but not limited to anti-viral medications, antibiotics, anti-fungal medications or anti-mold medications or anti-cancer medications, wherein the sound wave treatment improves the utility of these medications by increasing the amounts of these medications to the affected cells by increasing the short term and permanent blood supply to the cells and increasing the cellular communication to cause the body to aid in the fight against the foreign material. The treatment increases the permeability of the patient’s cell membranes allowing an increase in releasing anti-microbial peptides and inflow of the medications into the cells while increasing the blood supply toward the infection. The treatment can be provided prior to, during or after administering medications, chemotherapy, radiation or checkpoint inhibitors or any combination thereof. The dosages or strength of the medications can be reduced when used in combination with the treatment. The acoustic shock waves or pressure pulses can be focused or non-focused, convergent, divergent, planar or nearly planar, radial or spherical, shaped, linear or otherwise reflected or directed and are emitted by a generator or any mechanical device, wherein the generator or mechanical device is one of a radial, a spherical, a ballistic, a linear, a piezoelectric, or an electrohydraulic or electromagnetic generator. The treatment can be administered with or without cavitation.

In another embodiment, a method of treating a patient having cancer cells has the step of directing sound energy from one or more acoustic shock wave or pressure pulse treatments into the patient targeting the cancer cells in the patient’s blood to cause the patient’s immune system to identify the cancer cells as foreign objects and trigger a natural healing mechanism of the patient’s immune system to destroy the foreign objects. Wherein targeting the blood initiates cellular communication resulting in an unmasking of all cancer cells in the patient’s body by destroying a cloaking capability of the cancer cells which made the cancer cells indistinguishable from normal cells allowing the cancer cells to hide from the immune system prior to the one or more acoustic shock wave or pressure pulse treatments.

Treating the blood causes the cancer cells to secrete exosomes into extracellular spaces with MRNA that triggers a biologic cascade and cellular communication or signaling alerting the immune system to identify the cancer cells as foreign objects to target and destroy the cancer cells. The cellular communication or signaling is a secretion by the cancer cells to send exosomes outside of the cancer cells with proteins that the unmasking the cloaking capability of the cancer cells initiates causing the biologic cascade. During the acoustic shock wave or pressure pulse treatment no cancer cells are destroyed by the sound energy, however soon thereafter the unmasking the cancer cells allows the immune system to attack all cancer cells.

The method of this embodiment further can include treating a reflexology zone to reduce systemic inflammation to accelerate cancer recovery.

The treatment includes an improved long and short term blood supply, these mechanisms include both short and long term improvements in blood supply, an up regulation of anti-microbial peptides, especially peptide LL 37, a disruption of biofilms that protect these foreign objects, and an increase in cellular communication such that healthy cells identify these foreign objects as targets of the body’s natural defenses. This improved blood supply allows a body to deliver natural defenses and increases the supply of medications administered by a physician or other medications, a disruption of cellular membranes, increased cell membrane permeability, and an improvement in cellular communication causing the patient’s immune system to identify and attack the cancer cells and further causes recruiting or stimulating an increase in anti-microbial peptides.

The method further may include the step of administering one or more of medications, chemotherapy, radiation or checkpoint inhibitors to the patient including, but not limited to anti-viral medications, antibiotics, anti-fungal medications or anti-mold medications or anti-cancer medications, wherein the sound wave treatment improves the utility of these medications by increasing the amounts of these medications to the affected cells by increasing the short term and permanent blood supply to the cells and increasing the cellular communication to cause the body to aid in the fight against the foreign material.

The treatment increases the permeability of the patient’s cell membranes allowing an increase in releasing anti-microbial peptides and inflow of the medications into the cells while increasing the blood supply toward the infection. The treatment is provided prior to, during or after administering medications, chemotherapy, radiation or checkpoint inhibitors or any combination thereof. The dosages or strength of the medications can be reduced when used in combination with the treatment.

The acoustic shock waves or pressure pulses can be focused or non-focused, convergent, divergent, planar or nearly planar, radial or spherical, shaped, linear or otherwise reflected or directed and are emitted by a generator or any mechanical device, wherein the generator or mechanical device is one of a radial, a spherical, a ballistic, a linear, a piezoelectric, or an electrohydraulic or electromagnetic generator. The treatment can be administered with or without cavitation.

Treating the blood to initiate an immune response is achieved by targeting the heart where blood pools before pumping or by targeting the large vessels in the groin or even targeting the patient’s hands and/or feet to initiate the immune response to destroy circulating cancer and virus cells in the blood, outside of a tumor and get the same response as if targeting the tumor.

The method of treating blood further may have the steps of drawing blood into an IV bag and clamping it off from the patient; treating the blood with 1000 pulses thoroughly treating the blood with the acoustic shock waves or pressure pulses in a volume that cannot harm the patient when removed; and after treating the blood, returning the treated blood to the patient with another IV line and drawing a next portion until most of the blood in the patient has been treated, wherein the sound energy is in the range of .001 mJ/mm² and .30 mJ/mm².

### DEFINITIONS

“aerobic” living, active, or occurring only in the presence of oxygen.

“anaerobic” living, active, or occurring in the absence of free oxygen.

“apoptosis” is the biological process of controlled, programmed cell death, by means of which cells die by a process of condensation without the release of cell contents into the surrounding milieu.

A “curved emitter” is an emitter having a curved reflecting (or focusing) or emitting surface and includes, but is not limited to, emitters having ellipsoidal, parabolic, quasi parabolic (general paraboloid) or spherical reflector/reflecting or emitting elements. Curved emitters having a curved reflecting or focusing element generally produce waves having focused wave fronts, while curved emitters having a curved emitting surfaces generally produce wave having divergent wave fronts.

“cytoplasm” The part of a cell that contains the CYTOSOL and small structures excluding the CELL NUCLEUS; MITOCHONDRIA; and large VACUOLES.

“Divergent waves” in the context of the present invention are all waves which are not focused and are not plane or nearly plane. Divergent waves also include waves which only seem to have a focus or source from which the waves are transmitted. The wave fronts of divergent waves have divergent characteristics. Divergent waves can be created in many different ways, for example: A focused wave will become divergent once it has passed through the focal point. Spherical waves are also included in this definition of divergent waves and have wave fronts with divergent characteristics.

“extracorporeal” occurring or based outside the living body.

A “generalized paraboloid” according to the present invention is also a three-dimensional bowl. In two dimensions (in Cartesian coordinates, x and y) the formula yⁿ=2px [with n being ≠ 2, but being greater than about 1.2 and smaller than 2, or greater than 2 but smaller than about 2.8]. In a generalized paraboloid, the characteristics of the wave fronts created by electrodes located within the generalized paraboloid may be corrected by the selection of (p (-z,+z)), with z being a measure for the burn down of an electrode, and n, so that phenomena including, but not limited to, burn down of the tip of an electrode (-z,+z) and/or disturbances caused by diffraction at the aperture of the paraboloid are compensated for.

“lactate dehydrogenase (LDH)” A tetrameric enzyme that, along with the coenzyme NAD+, catalyzes the interconversion of lactate and pyruvate. In vertebrates, genes for three different subunits (LDH-A, LDH-B and LDH-C) exist.

“mitochondria” Semiautonomous, self-reproducing organelles that occur in the cytoplasm of all cells of most, but not all, eukaryotes. Each mitochondrion is surrounded by a double limiting membrane. The inner membrane is highly invaginated, and its projections are called cristae. Mitochondria are the sites of the reactions of oxidative phosphorylation, which result in the formation of ATP. They contain distinctive RIBOSOMES, transfer RNAs (RNA, TRANSFER); AMINO ACYL T RNA SYNTHETASES; and elongation and termination factors. Mitochondria depend upon genes within the nucleus of the cells in which they reside for many essential messenger RNAs (RNA, MESSENGER). Mitochondria are believed to have arisen from aerobic bacteria that established a symbiotic relationship with primitive protoeukaryotes.

“necrosis” A pathological process caused by the progressive degradative action of enzymes that is generally associated with severe cellular trauma. It is characterized by mitochondrial swelling, nuclear flocculation, uncontrolled cell lysis, and ultimately CELL DEATH.

A “paraboloid” according to the present invention is a three-dimensional reflecting bowl. In two dimensions (in Cartesian coordinates, x and y) the formula y²=2px, wherein p/2 is the distance of the focal point of the paraboloid from its apex, defines the paraboloid. Rotation of the two-dimensional figure defined by this formula around its longitudinal axis generates a de facto paraboloid.

“phagocytosis” The engulfing of microorganisms, other cells, and foreign particles by phagocytic cells.

“Plane waves” are sometimes also called flat or even waves. Their wave fronts have plane characteristics (also called even or parallel characteristics). The amplitude in a wave front is constant and the “curvature” is flat (that is why these waves are sometimes called flat waves). Plane waves do not have a focus to which their fronts move (focused) or from which the fronts are emitted (divergent). “Nearly plane waves” also do not have a focus to which their fronts move (focused) or from which the fronts are emitted (divergent). The amplitude of their wave fronts (having “nearly plane” characteristics) is approximating the constancy of plain waves. “Nearly plane” waves can be emitted by generators having pressure pulse/ shock wave generating elements with flat emitters or curved emitters. Curved emitters may comprise a generalized paraboloid that allows waves having nearly plane characteristics to be emitted.

A “pressure pulse” according to the present invention is an acoustic pulse which includes several cycles of positive and negative pressure. The amplitude of the positive part of such a cycle should be above about 0.1 MPa and its time duration is from below a microsecond to about a second. Rise times of the positive part of the first pressure cycle may be in the range of nano-seconds (ns) up to some milli-seconds (ms). Very fast pressure pulses are called shock waves. Shock waves used in medical applications do have amplitudes above 0.1 MPa and rise times of the amplitude can be below 1000 ns, preferably at or below 100 ns. The duration of a shock wave is typically below 1-3 micro-seconds (µs) for the positive part of a cycle and typically above some micro-seconds for the negative part of a cycle.

Prostate-specific antigen, also known as gamma-seminoprotein or kallikrein-3, is a glycoprotein enzyme encoded in humans by the KLK3 gene. PSA is a member of the kallikrein-related peptidase family and is secreted by the epithelial cells of the prostate gland. PSA is present in small quantities in the serum of men with healthy prostates, but is often elevated in the presence of prostate cancer or other prostate disorders. PSA is not a unique indicator of prostate cancer, but may also detect prostatitis or benign prostatic hyperplasia. The PSA test is a blood test used primarily to screen for prostate cancer. The test measures the amount of prostate-specific antigen (PSA) in your blood. PSA is a protein produced by both cancerous and noncancerous tissue in the prostate, a small gland that sits below the bladder in men.

“Reflexology zone” as used herein means an area or pressure point on the feet or hands that are access pathways to every organ, gland, muscle, etc. These pathways between pressure points and other parts of the body are thought to be connected via the nervous system and that a neurological relationship exists between the skin and the internal organs, and that the whole nervous system adjusts to a stimulus. According to reflexology theory, application of pressure to feet, hands, or ears sends a calming message from the peripheral nerves in these extremities to the central nervous system, which in turn signals the body to adjust the tension level. This enhances overall relaxation, removes stress, brings internal organs and their systems into a state of optimum functioning, and increases blood supply which brings additional oxygen and nutrients to cells and enhances waste removal. It positively affects the circulatory, respiratory, endocrine, immune, and neuropeptide systems in the body.

“Sound wave”: As used herein means any wave generated as sound moving in or through a medium. “Sound waves” include acoustic shock waves, pressure pulses, of a variety of types, or asymmetric sound waves including sub sonic, ultra sound (a sinusoidal wave), radial, spherical, focused, unfocused, convergent, divergent, planar, near planar, Linear, or combinations thereof.

“Shock Wave”: As used herein is defined by Camilo Perez, Hong Chen, and Thomas J. Matula; Center for Industrial and Medical Ultrasound, Applied Physics Laboratory, University of Washington, 1013 NE 40th Street, Seattle, Washington 98105; Maria Karzova and Vera A. Khokhlovab; Department of Acoustics, Faculty of Physics, Moscow State University, Moscow 119991, Russia; (Received 9 Oct. 2012; revised 16 Apr. 2013; accepted 1 May 2013) in their publication, “Acoustic field characterization of the Duolith: Measurements and modeling of a clinical shock wave therapy device”; incorporated by reference herein in its entirety.

Wave energy or energy flux density: the measurement of energy flux density is defined as the energy directed toward the target or region being treated. This is not energy at the gap between electrodes, but rather the energy transmitted toward the patient’s tissue through the skin.

Waves/wave fronts described as being “focused” or “having focusing characteristics” means in the context of the present invention that the respective waves or wave fronts are traveling and increase their amplitude in direction of the focal point. Per definition the energy of the wave will be at a maximum in the focal point or, if there is a focal shift in this point, the energy is at a maximum near the geometrical focal point. Both the maximum energy and the maximal pressure amplitude may be used to define the focal point.

### BRIEF DESCRIPTION OF THE DRAWINGS

The invention will be described by way of example and with reference to the accompanying drawings in which:

FIG. 1 is a simplified depiction of a pressure pulse / shock wave (PP/SW) generator with focusing wave characteristics.

FIG. 2 is a simplified depiction of a pressure pulse / shock wave generator with plane wave characteristics.

FIG. 3 is a simplified depiction of a pressure pulse / shock wave generator with divergent wave characteristics.

FIG. 4 is a simplified depiction of a pressure pulse / shock wave generator connected to a control/power supply unit.

FIG. 5 is a graph showing an exemplary ultrasound wave pattern.

FIG. 6 is a graph of an exemplary acoustic shock wave pattern.

FIG. 7 shows a patient’s blood being treated in a bag.

FIG. 8, shows a patient’s blood being treated through a bag and an IV.

FIG. 9 shows the shock wave generator device directed at a reflexology zone on a foot of a patient.

FIG. 10 shows the shock wave generator device directed at a reflexology zone on a hand of a patient.

FIG. 11 shows a schematic view showing general reflexology locations of the foot in the human body.

FIG. 12 shows a schematic view showing general reflexology locations of the hand in the human body.

FIG. 13 shows a patient being treated in an area having a tumor.

### DETAILED DESCRIPTION OF THE INVENTION

Sound waves can treat cancerous tumors. The treatment of the tumor stimulates the body to identify the tumor and to destroy the tumor utilizing a body’s own natural defenses. Tumors are often Trojan horses of cancer cells confusing the body as to the cell’s identity. Most importantly, when a tumor is targeted and eventually destroyed by sound waves utilizing a body’s own natural defenses, the body will seek out all other cancer cells of this type and destroy them as well, essentially permanently vaccinating the body against this type of cancer.

Targeting at least one tumor directly to initiate a cascade effect allows the immune system to identify other similar cells in the body and attack them. Reflexology treatments can be added to reduce systemic inflammation to accelerate cancer recovery.

Shocking the tumor initiates cellular communication resulting in the targeting of all cancer cells in the body. It destroys the trojan horse capabilities of cancer cells. This causes cancer cells to send exosomes into extracellular spaces with MRNA that triggers the biologic cascade effect by cellular communication telling total immune system to target cancer cells.

This use of acoustic shock waves or pressure pulses does not destroy cancer cells. It does not damage cancer cells, but rather causes cancer cells to send exosomes outside of the cell with proteins that initiate the trojan killing cascade. Immediately after treatment there are no destroyed cells, however soon thereafter the immune system attacks all cancer cells. This is a huge difference from prior art approaches where they require damage to cancer cells by fragmenting. The present invention uses sheering forces to open exosome and other cellular channels enhancing cellular communication without fragmenting the cancer cells.

The present invention concerns treating not only cancer but virus cells that share many of the characteristics of cancer cells that allow them to be damaged/destroyed by SWT. This treatment makes these cells susceptible to the normal immune response and immunizing the body to seek out and destroy other cancer and virus cells. It is believed that it is not necessary to actually target a tumor to get this immune response/inoculation.

FIG. 13 illustrates a treatment targeting cancer cells 100 in a tumor in a patient’s P back or spine area 101 through the patient’s skin Ps. The exemplary treatment shows the patient P laying on a table T being treated with an adjustable fixed shock wave device 44 consisting of an applicator head 43, flexible hose 42 and shock wave or pressure pulse generator 41. As shown, the shock wave applicator head 43 is brought into contact with the skin Ps preferably using an acoustic gel to enhance the transmission of the shock waves 200 through the body down to the cancer cells 100 in the area 101. The shock wave applicator head 43 can be attached rigidly to a fixture or stand 44, as illustrated, or alternatively can be hand held and manipulated across the skin Ps to drive the shock waves 200 in the direction the shock wave head 43 is pointed to activate a response.

As an example for blood cancers, if blood is treated long enough and frequently enough, the circulating blood cancer cells can be injured or destroyed initiating this same immune response. It is only a question of dosage. For instance, if the heart is targeted where blood pools before pumping, this could treat all of the blood in the body and destroy enough cancer cells to initiate the immune response. One could also target the large vessels in the groin or even the hands and feet if treated long enough. One can destroy circulating cancer and virus cells in the blood, outside of a tumor and get the same response as if targeting a tumor.

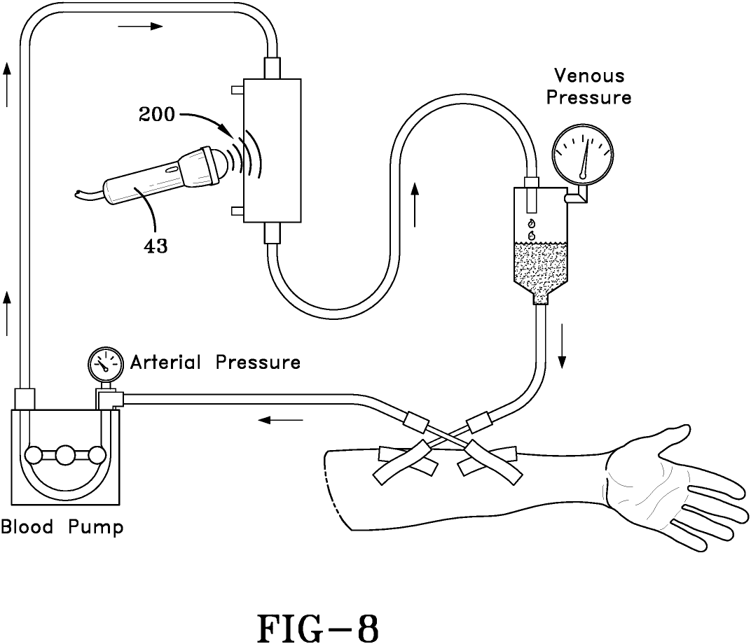

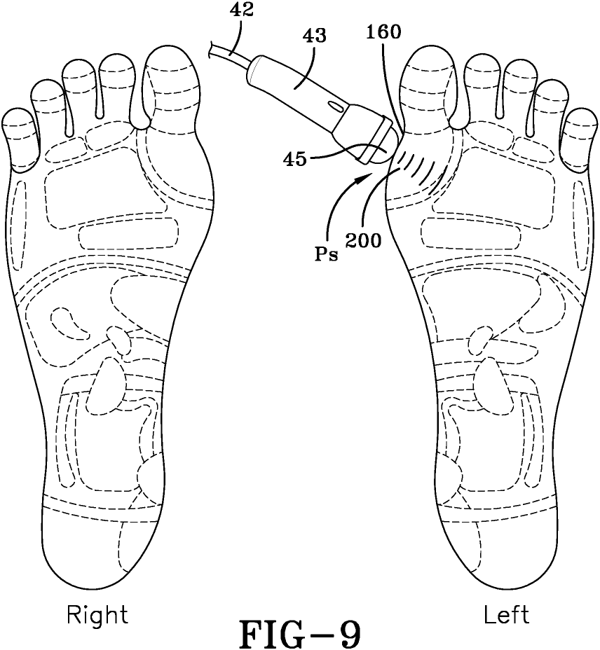

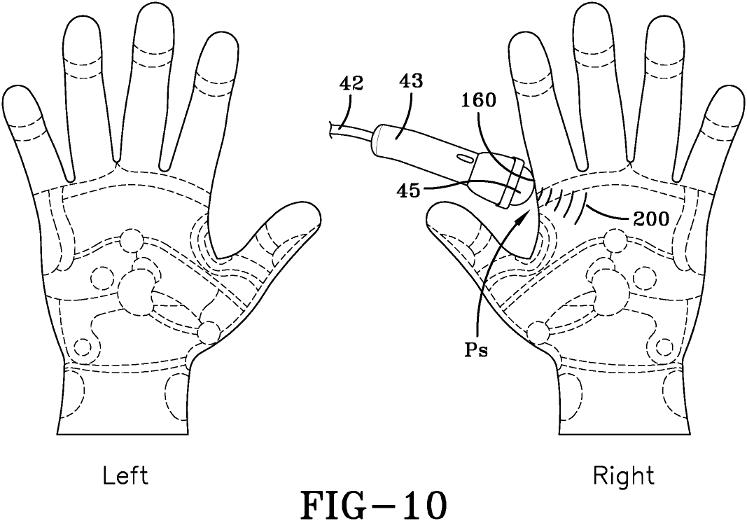

One treatment, as shown in FIGS. 7 and 8, is to drain blood having cancer cells 100 into an IV bag 202 and clamp it off from the patient’s body in both directions and treat it with 1000 pulses thoroughly treating this portion of blood with acoustic shock waves or pressure pulses 200 from a shock wave applicator 43. Using a volume of blood that will not harm the patient when removed. As shown the blood can be treated separately in a bag 202 or in the tubing or bag attached to a patient. After this blood has been treated, it is returned to the body with another IV line and the next portion of blood is drawn until most of the blood in the body has been treated. The known ranges of energy that can destroy or injure cancer and virus cells is in the range of 0.001 mJ/mm² and 0.30 mJ/mm².

Ideally the response can be initiated with only 1 treatment however with the inherent uncertainties it could take as many as 10 treatments.

Blood cancer and viruses are the most obvious targets, however it is known that patients with tumors actually have circulating cancer cells in the blood that could be targeted to initiate the response.

In the extracorporeal shock wave or pressure pulse method of treating a cancer patient, the administered shock waves or pressure pulses are directed to a treatment location or target site on the anatomy. In this invention, the term target site refers to either a location near the source of the tumor, the blood or to a reflexology location for a specific orthopedic bone structure, nerve, gland and the tissue of the hand or foot at the desired reflexology zone or region being in the path of the shock wave applicator. As used herein, “near” recognizes that the emitted shock waves or pressure pulses are transmitted through the skin and subcutaneous tissue directed toward the treatment location, preferably at or in close proximity to the treatment location or site. The patient is placed in a convenient orientation to permit the source of the emitted waves to most directly send the waves to the target site to initiate shock wave stimulation of the target area. Assuming the target area is within a projected area of the wave transmission, a single transmission dosage of wave energy may be used. The transmission dosage can be from a few seconds to 20 minutes or more dependent on the condition. Preferably the waves are generated from an unfocused or focused source. The unfocused waves can be divergent or near planar and having a low-pressure amplitude and density in the range of 0.00001 mJ/mm² to 1.0 mJ/mm² or less, most typically below 0.2 mJ/mm². These are typically generated by spherical or radial wave generators, ballistic or electrohydraulic wave or piezoelectric shock wave generators. The focused source can use a focused beam of waves or can optionally use a diffusing lens or have a far-sight focus to minimize if not eliminate having the localized focus zone within the tissue. Preferably the focused shock waves are used at a similarly effective low energy transmission or alternatively can be at higher energy but wherein the tissue target site is disposed pre-convergence inward of the geometric focal point of the emitted wave transmission. Understanding the higher the energy used, the more sensation of pain the patient may experience. In these cases, cavitation can and often does occur as well as bruising and come cell damage.

These shock wave or pressure pulse energy transmissions are effective in stimulating a cellular response and in some cases, such as unfocused low energy, and even low energy focused emissions can be accomplished without creating the localized hemorrhaging caused by rupturing cavitation bubbles in the tissue of the target site. This effectively ensures the patient does not have to experience the sensation of pain so common in the higher energy focused wave forms having a focal point at or within the targeted treatment site. Higher energy acoustic shock waves or pressure pulses including focused waves can be used.

The target site within the body may be such that the patient or the generating source must be reoriented relative to the site and a second, third or more treatment dosage can be administered. At a low energy, the common problem of localized hemorrhaging is reduced making it more practical to administer multiple dosages of waves from various orientations to further optimize the treatment and cellular stimulation of the target site. Alternatively, focused high energy multiple treatments can be equally effective, but with induced pain and more discomfort to the patient. The use of low energy focused or un-focused waves at the target site enables multiple sequential treatments. Alternatively, the wave source generators may be deployed in an array wherein the subject patient is effectively enveloped or surrounded by a plurality of low energy wave source generators which can be simultaneously bombarding the target site from multiple directions. Such arrays include linear type devices.

The goal in such treatments is to provide 100 to 3000 acoustic shock waves or pressure pulses. Typically, at a voltage of 14 kV to 28 kV across a spark gap generator in a single treatment preferably or one or more adjuvant treatments by targeting the site directly by impinging the emitted waves toward the infection or indirectly on the desired reflexology target.

The present method, in many cases, does not rely on precise site location per se. The physician’s general understanding of the anatomy of the patient should be sufficient to locate a desirable direct path or using a reflexology target site to indirectly attack the infection be treated. The treated area can withstand a far greater number of shock waves based on the selected energy level being emitted. For example, at very low energy levels the stimulation exposure can be provided over prolonged periods as much as 20 minutes if so desired. At higher energy levels the treatment duration can be shortened to less than a minute, less than a second if so desired. The selected treatment dosage can include the avoidance or minimization of cell hemorrhaging and other kinds of damage to the cells or tissue while still providing a stimulating cellular release activation of upregulation of the antimicrobial peptide LL37, a protein that can bind with RNA to destroy the infection, and also VEGF and other growth factors and can also be used to modulate and regulate hormonal secretions from a specific targeted gland by emitting waves to a desired direct path or indirectly selected using a targeted reflexology zone. In other cases where the precise location must be known, the use of an applicator acoustic wave emission is directed by an ultrasound image, preferably the applicator has a software program coupled to the imaging device to allow the doctor to visualize the area being treated. The applicator can be hand held or manipulated in a fixture, if so desired, in either way the doctor can see the reflexology zone for any gland to be stimulated and the selected reflexology zone reflects the path of the wave transmission to modulate that bone structure, nerve or gland.

A key advantage of the present inventive methodology is that it is complimentary to conventional medical procedures. In the case of any other procedure, the area of the patient can be post operatively bombarded with sound waves to stimulate cellular release of healing agents and growth factors. Most preferably such patients may be provided more than one such ESWT treatment with an intervening dwell time for cellular relaxation prior to secondary and tertiary treatments.

The underlying principle of these sound wave therapy methods is to stimulate the body’s own natural healing capability. This is accomplished by deploying shock waves to stimulate strong cells in the tissue to activate a variety of responses, more particularly those that reduce inflammation and stop the infection. The sound waves including acoustic shock waves or pressure pulses transmit or trigger what appears to be a cellular communication throughout the entire anatomical structure, this activates a generalized cellular response at the treatment or target site, in particular, but more interestingly a systemic response in areas more removed from the wave form pattern. This is believed to be one of the reasons molecular stimulation can be conducted at threshold energies heretofore believed to be well below those commonly accepted as required. Accordingly, not only can the energy intensity be reduced but also the number of applied shock wave impulses can be lowered from several thousand to as few as one or more pulses and still yield a beneficial stimulating response if desired.

The biological model motivated the design of sources with low pressure amplitudes and energy densities. First: spherical waves generated between two tips of an electrode; and second: nearly even waves generated by generated by generalized parabolic reflectors. Third: divergent shock front characteristics are generated by an ellipsoid behind F2. Unfocused sources are preferably designed for extended two dimensional areas/volumes like skin. The unfocused sources can provide a divergent wave pattern or a nearly planar wave pattern and can be used in isolation or in combination with focused wave patterns yielding to an improved therapeutic treatment capability that is non-invasive with few if any disadvantageous contraindications. Alternatively, a focused wave emitting treatment may be used wherein the focal point extends to the desired reflexology zone or target site. In any event, the beam of acoustic waves transmitted needs to project in a large enough reflexology zone or area to stimulate or modulate the cells near the infection of cancerous cells.

In one embodiment, the method of treatment has the steps of, locating a reflexology treatment site or zone, generating either focused shock waves or unfocused shock waves, of directing these shock waves to the treatment site; and applying a sufficient number of these shock waves to induce activation of one or more growth factor or anti-microbial peptides like LL37, thereby inducing or accelerating a modulated adjustment to induce the host cells to attack the cancer cells.

The shock waves can be of a low peak pressure amplitude and density. Typically, the energy density values range as low as 0.000001 mJ/ mm² and having a high end energy density of below 1.0 mJ/ mm², preferably 0.40 mJ/mm² or less, more preferably 0.20 mJ/ mm² or less. The peak pressure amplitude of the positive part of the cycle should be above 1.0 and its duration is below 1-3 microseconds.

The treatment depth can vary from the surface to the full depth of the human or animal torso and the treatment site can be defined by a much larger treatment area. The above methodology is particularly well suited for surface as well as sub-surface soft tissue treatments.

As used herein, a sound wave treatment is defined as the transmission of emitted shock waves or pressure pulses to a treatment location or target site of a patient. An exemplary sound wave treatment protocol could have emitted shock waves in a broad range of 0.01 mJ/mm² to 3.0 mJ/mm² and 200-2500 pulses per treatment with a treatment schedule of 1-3 weekly treatments until symptoms reduce. This can be repeated as symptoms reoccur or continue weekly as a preventative. The post medical treatment is beneficial as a pain suppressor and reduces the need for pain medications and allows less addictive medications to be used to prevent addiction. In other treatment protocols, the emitted shock waves or pressure pulses can employ as few as 1 to as high as 100,000 pulses per treatment.

The above methodology is valuable in generation of tissue, vascularization and may be used in combination with stem cell therapies as well as regeneration of tissue and vascularization.

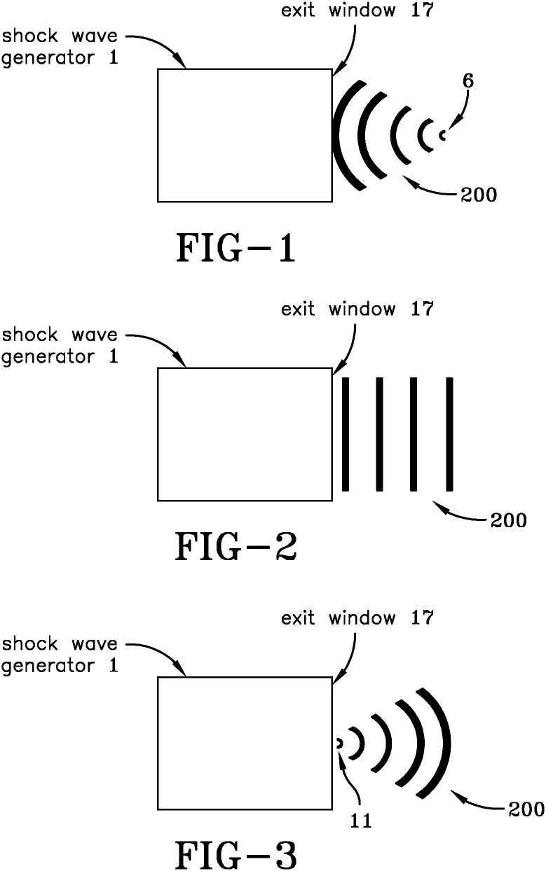

The following invention description first provides a detailed explanation of acoustic shock waves or pressure pulses, as illustrated in FIGS. 1 - 6. As used herein an acoustic shock wave or pressure pulse is an asymmetric wave with an exceptionally rapid peak rise time and slower return time from the peak amplitude. Historically, these acoustic shock waves or pressure pulses were first used medically to destroy kidney stones. The wave patterns were directed to a focal point at a relatively high energy to blast the concrements into small urinary tract passable fragments.

A whole class of acoustic shock waves or pressure pulses for medical treatments were later discovered that employed low energy acoustic shock waves or pressure pulses. These low energy acoustic shock waves or pressure pulses maintained the asymmetric wave profile, but at much lower energies.

These low energy acoustic shock waves or pressure pulses advantageously could stimulate a substance without requiring a focused beam. The advantage of such an unfocused beam was the acoustic wave could be directed to pass through tissue without causing any cell rupturing which would be evidenced by a lack of a hematoma or bruising. This use of unfocused, low energy acoustic shock waves or pressure pulses provided an ability to treat a large volume of tissue virtually painlessly. Furthermore, the acoustic energy caused a short duration anesthetic sensation that effectively numbs the patient’s pain over a period of days with a prolonged reduction in pain thereafter.

The use of low energy acoustic shock waves or pressure pulses that employ a focused beam has been spurred on as a viable alternative to the unfocused low energy shock waves because the focal point being of a small zone of energy has little or a small region of cell damage as the remaining portions of the wave pattern can provide a stimulating effect similar to the unfocused shock waves. Basically, the effect is the same with the users of focused waves achieving the benefits of the unfocused waves, but with a focal point of peak energy in a tiny localised region. So, for purposes of the present invention, the use of “soft waves” those defined by low energy beams will be applicable to both focused and unfocused beams of acoustic shock waves or pressure pulses.

With reference to FIGS. 1 - 3, a variety of schematic views of acoustic shock waves or pressure pulses are described. The following description of the proper amplitude and pressure pulse intensities of the shock waves are provided along with a description of how the shock waves actually function. For the purpose of describing, the shock waves were used as exemplary and are intended to include all of the wave patterns discussed in the figures as possible treatment patterns for treating sensitive gum tissue.

FIG. 1 is a simplified depiction of a pressure pulse / shock wave (PP/SW) generator, such as a shock wave head, showing focusing characteristics of transmitted acoustic pressure pulses. Numeral 1 indicates the position of a generalized pressure pulse generator, which generates the pressure pulse and, via a focusing element, focuses it outside the housing to treat diseases. The affected tissue or organ is generally located in or near the focal point which is located in or near position 6. At position 17 a water cushion or any other kind of exit window for the acoustical energy is located.

FIG. 2 is a simplified depiction of a pressure pulse / shock wave generator, such as a shock wave head, with plane wave characteristics. Numeral 1 indicates the position of a pressure pulse generator according to the present invention, which generates a pressure pulse which is leaving the housing at the position 17, which may be a water cushion or any other kind of exit window. Somewhat even, also referred to herein as “disturbed”, wave characteristics can be generated, in case a paraboloid is used as a reflecting element, with a point source (e.g. electrode) that is located in the focal point of the paraboloid. The waves will be transmitted into the patient’s body via a coupling media such as, e.g., ultrasound gel or oil and their amplitudes will be attenuated with increasing distance from the exit window 17.

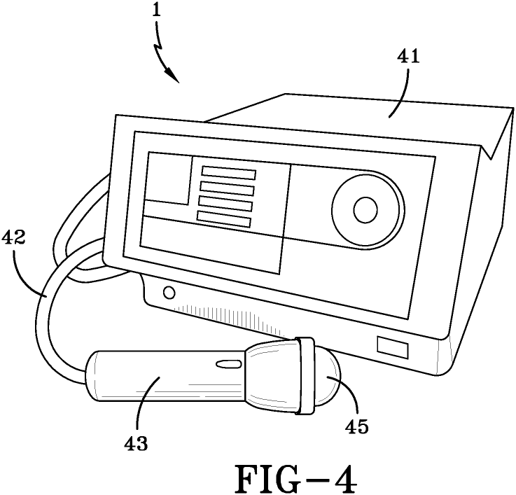

FIG. 3 is a simplified depiction of a pressure pulse shock wave generator (shock wave head) with divergent wave characteristics. The divergent wave fronts may be leaving the exit window 17 at point 11 where the amplitude of the wave front is very high. This point 17 could be regarded as the source point for the pressure pulses. In FIG. 3, the pressure pulse source may be a point source, that is, the pressure pulse may be generated by an electrical discharge of an electrode under water between electrode tips. However, the pressure pulse may also be generated, for example, by an explosion, referred to as a ballistic pressure pulse. The divergent characteristics of the wave front may be a consequence of the mechanical setup.

With reference to FIG. 4, an exemplary acoustic shock wave apparatus 1 is illustrated. The shock wave apparatus 1 has a generator 41 connected by a flexible hose with fluid conduits extending from the shock wave generator 41 to an applicator 43 which transmits the acoustic waves when coupled to the skin by using a fluid or acoustic gel. The applicator 43 as illustrated has a body that enables a technician to hold the applicator 43 and as illustrated this applicator is an electrohydraulic that is filled with fluid to facilitate the transmission of the shock waves. The fluid expands a flexible membrane in such a fashion that the membrane extends outwardly in a balloon shape fashion as illustrated in FIG. 4. As shown, this type of applicator 43 has a hydraulic spark generator using either focused or unfocused shock waves, preferably in a low energy level, less than the range of 0.01 mJ/mm² to 0.3 mJ/mm². The flexible hose 42 is connected to a fluid supply that fills the applicator 43 and expands the flexible membrane when filled. Alternatively, a ballistic, piezoelectric or spherical acoustic shock wave device can be used to generate the desired waves.

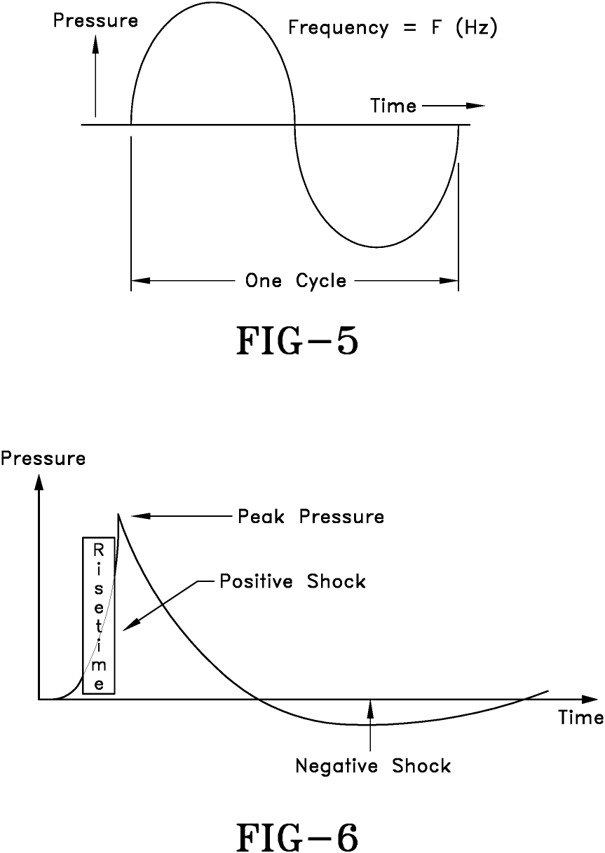

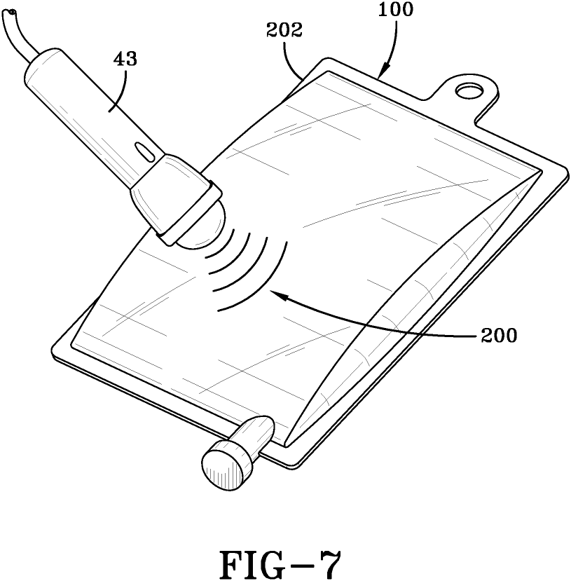

The ultrasonic wave pattern shown in FIG. 5 is contrasted to an asymmetric acoustic wave pattern which is illustrated in FIG. 6. As shown, ultrasound waves are symmetrical having the positive rise time equal to the negative in a sinusoidal wave form. These ultrasound waves generate heat in the tissue and are accordingly believed not suitable for use on organs or sensitive tissue.

This apparatus, in certain embodiments, may be adjusted/modified/or the complete shock wave head or part of it may be exchanged so that the desired and/or optimal acoustic profile such as one having wave fronts with focused, planar, nearly plane, convergent or divergent characteristics can be chosen.

A change of the wave front characteristics may, for example, be achieved by changing the distance of the exit acoustic window relative to the reflector, by changing the reflector geometry, by introducing certain lenses or by removing elements such as lenses that modify the waves produced by a pressure pulse/shock wave generating element. Exemplary pressure pulse/shock wave sources that can, for example, be exchanged for each other to allow an apparatus to generate waves having different wave front characteristics are described in detail below.

In one embodiment, mechanical elements that are exchanged to achieve a change in wave front characteristics include the primary pressure pulse generating element, the focusing element, the reflecting element, the housing and the membrane. In another embodiment, the mechanical elements further include a closed fluid volume within the housing in which the pressure pulse is formed and transmitted through the exit window.

In one embodiment, the apparatus of the present invention is used in combination therapy. Here, the characteristics of waves emitted by the apparatus are switched from, for example, focused to divergent or from divergent with lower energy density to divergent with higher energy density. Thus, effects of a pressure pulse treatment can be optimized by using waves having different characteristics and/or energy densities, respectively.

While the above described universal toolbox of the various types of acoustic shock waves or pressure pulses and types of shock wave generating heads provides versatility, the person skilled in the art will appreciate that apparatuses that produce acoustic shock waves or pressure pulses having, for one example, nearly plane characteristics, are less mechanically demanding and fulfil the requirements of many users.

As the person skilled in the art will also appreciate that embodiments shown in the drawings are independent of the generation principle and thus are valid for not only electro-hydraulic shock wave generation but also for, but not limited to, PP/SW generation based on electromagnetic, piezoceramic and ballistic principles. The pressure pulse generators may, in certain embodiments, be equipped with a water cushion that houses water which defines the path of pressure pulse waves that is, through which those waves are transmitted. In a preferred embodiment, a patient is coupled via ultrasound gel or oil to the acoustic exit window (17), which can, for example, be an acoustic transparent membrane, a water cushion, a plastic plate or a metal plate.

These shock wave or pressure pulse energy transmissions are effective in stimulating a cellular response and can be accomplished without creating the cavitation bubbles in the tissue of the target site when employed in other than high energy focused transmissions. This effectively ensures the tissue or organ does not have to experience the sensation of hemorrhaging so common in the higher energy focused wave forms having a focal point at or within the targeted treatment site.

The present method may need precise site location and can be used in combination with such known devices as ultrasound, cat-scan or x-ray imaging if needed. The physician’s general understanding of the anatomy of the patient may be sufficient to locate the target area to be treated. This is particularly true when the exposed tissue or portion of the infected body or organ is visually within the surgeon’s line of sight and this permits the lens or cover of the emitting shock wave source to impinge on the affected organ or tissue directly or through a transmission enhancing gel, water or fluid medium during the pressure pulse or shock wave treatment. The treated area can withstand a far greater number of shock waves based on the selected energy level being emitted. For example, at very low energy levels the stimulation exposure can be provided over prolonged periods as much as 20 minutes if so desired. At higher energy levels the treatment duration can be shortened to less than a minute, less than a second if so desired. The limiting factor in the selected treatment dosage is to provide a stimulating stem cell activation or a cellular release or activation of the LL37 protein and VEGF and other growth factors while simultaneously germicidally attacking the infection barrier and underlying colony of microorganisms.

The underlying principle of these pressure pulse or shock wave therapy methods is to attack the at least one tumor of cancerous cells directly which allows the remaining non-targeted cancer cells to be identified and destroyed by the patient’s own immune system by stimulating the body’s own natural healing capability. This is accomplished by deploying shock waves to stimulate strong cells in the surrounding tissue to activate a variety of responses. The acoustic shock waves transmit or trigger what appears to be a cellular communication throughout the entire anatomical structure, this activates a generalized cellular response at the treatment site, in particular, but more interestingly a systemic response in areas more removed from the wave form pattern. This is believed to be one of the reasons molecular stimulation can be conducted at threshold energies heretofore believed to be well below those commonly accepted as required. Accordingly, not only can the energy intensity be reduced in some cases, but also the number of applied shock wave impulses can be lowered from several thousand to as few as one or more pulses and still yield a beneficial stimulating response. The key is to provide at least a sufficient amount of energy to weaken the protective outer barrier or shield commonly found in cancerous cells. This weakening increases the permeability of the membrane without any fracture or opening, but rather causes a secretion of proteins that unmasks and exposes the underlying colony of cancer cells.

The use of shock waves as described above achieved biological response within the cells and there appears to be a commonality in the fact that otherwise dormant cells within the tissue appear to be activated making the cell membranes more permeable to release anti-microbial peptides and absorb medications to attack infections which leads to the remarkable ability of the targeted organ or tissue to generate new growth or to regenerate weakened vascular networks increasing blood supply.

FIG. 9 is a perspective view of a foot of a patient whose reflexology zone or target 100 is being treated. A shock wave applicator head 43 is brought into contact with the skin Ps preferably an acoustic gel is used to enhance the transmission of the shock waves 200 through the skin Ps. The shock wave applicator head 43 can be hand held and manipulated across the skin Ps to drive the shock waves 200 in the direction the shock wave head 43 is zoned to activate a stimulating response through the reflexology zone 100. As illustrated, the device shown is an electrohydraulic acoustic shock wave generator, however, other devices that generate acoustic shock waves or pressure pulses can be used. Ultrasonic devices may be considered, but there is no data to support a sinusoidal wave form would work and therefore not considered as effective as the asymmetric wave generators. The acoustic shock waves or pressure pulses activate a cellular response within the reflexology treatment site. This response or stimulation causes an increase of nitric oxide and a release of a variety of growth factors such as VEGF and a release of anti-microbial peptides like LL37. As shown, the flexible membrane is protruding outward and the applicator 43 has been filled with fluid, the transmission or emission of acoustic shock waves or pressure pulses 200 is directed towards the reflexology zone 100. In order to accomplish a good transmission, it is important the flexible membrane be pressed against the patient’s skin Ps and as indicated coupling gels may be used. The zone 100, as illustrated, is the reflexology zone for a bone structure which is a region of the foot located along an outside arch of each foot. By transmitting the shock waves 200 to the zone 100, is it believed that a modulation of the pain near the bone structure can be made. This modulation or adjustment can be achieved by transmitting the acoustic waves 200 at low energy directly onto the zone 100. It is believed that a single treatment of the zone 100 will achieve the desired modulation. However, repeated treatments may be administered to help maintain and control this reduced pain level. Having achieved a scheduled pattern of treatments, it is possible to achieve regulation of pain without the use of drugs or other stimulants.

With reference to FIG. 10, a view of a hand of a patient whose reflexology zone 100 is being treated with acoustic shock waves or pressure pulses 200 is illustrated. In this illustration, it is important to note that the applicator 43 presses against the skin Ps of the hand in the reflexology zone 100 for the pancreas which is a region of the right hand in the fatty part below the index finger and a region of the left hand below the middle finger close to the wrist.

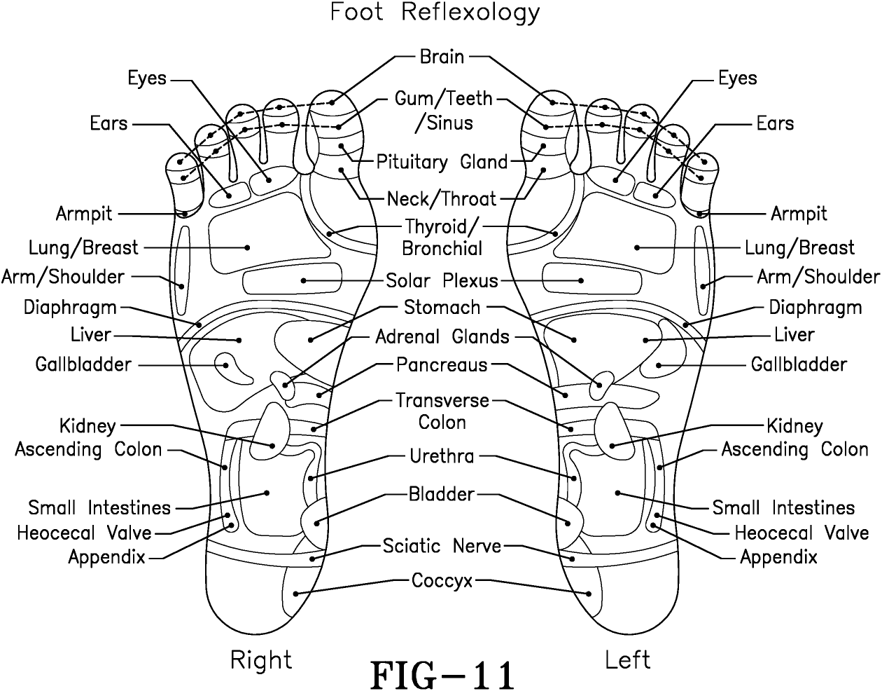

With reference to FIG. 11, a reflexology foot charts are shown detailing the various zones that correspond to organs, nerves, bones or glands of the body.

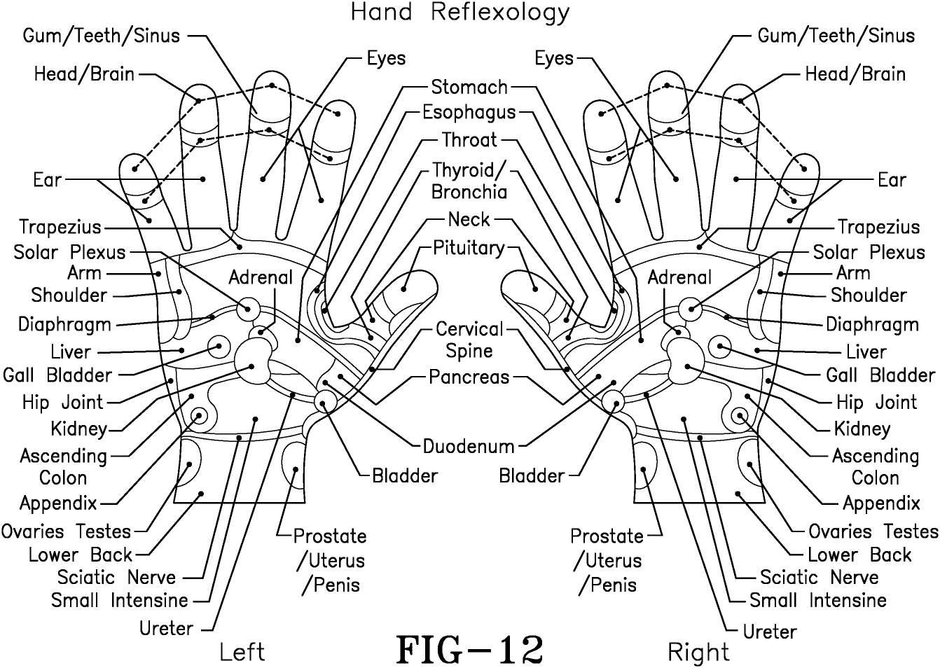

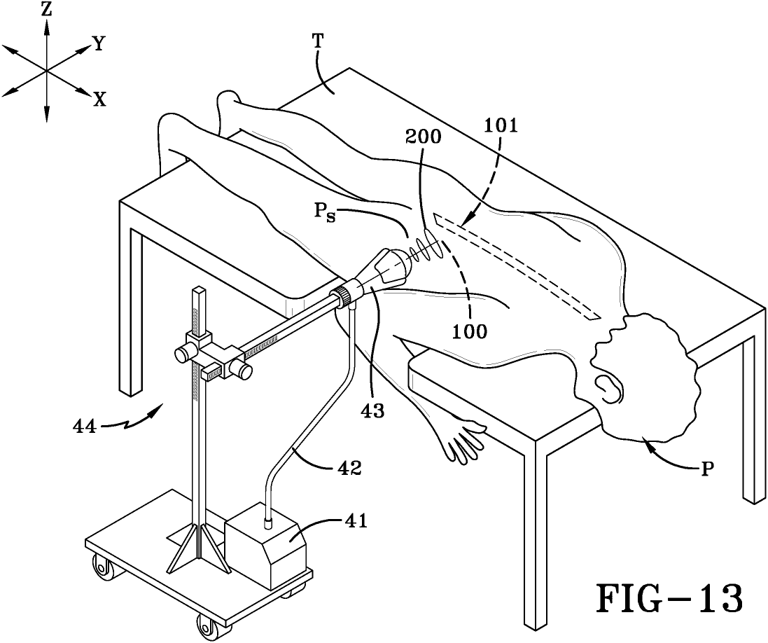

With reference to FIG. 12, a reflexology hand chart is shown detailing the various zones that correspond to organs, nerves, bones or glands of the body.

The transmission of the shock waves 200 can be of a low energy density of 0.2 mJ/mm² whether using focused or unfocused shock waves. The acoustic shock waves or pressure pulses pulse rapidly through the cells penetrating the cell membrane extremely rapidly due to the rapid rise to peak time and pass through exiting slower due to the slower return from peak amplitude. This asymmetric wave pattern rapidly compresses each cell on entry and slow decompresses the cell as it exits. This effective squeezing of each cell is believed to cause the release of growth factors such as VEGF and others and also creates nitric oxide, all beneficial to new blood vessel formation. This occurs as a transmission across the cell membranes without rupturing the native cells.

Furthermore, such acoustic shock wave forms can be used in combination with drugs, chemical treatments, irradiation therapy or even physical therapy and when so combined the stimulated cells will more rapidly assist the body’s natural healing response and thus overcomes the otherwise potentially tissue damaging effects of these complimentary procedures.

The present invention provides an apparatus for an effective treatment of indications, which benefit from high or low energy pressure pulse/ shock waves having focused or unfocused, nearly plane, convergent or even divergent characteristics. With an unfocused wave having nearly plane, plane, convergent wave characteristic or even divergent wave characteristics, the energy density of the wave may be or may be adjusted to be so low that side effects including pain are very minor or even do not exist at all.

In certain embodiments, the apparatus of the present invention is able to produce waves having energy density values that are below 0.1 mJ/mm² or even as low as 0.000001 mJ/mm². In a preferred embodiment, those low end values range between 0.1 - 0.001 mJ/mm². With these low energy densities, side effects are reduced and the dose application is much more uniform. Additionally, the possibility of harming surface tissue is reduced when using an apparatus of the present invention that generates unfocused waves having planar, nearly plane, convergent or divergent characteristics and larger transmission areas compared to apparatuses using a focused shock wave source that need to be moved around to cover the affected area. The apparatus of the present invention also may allow the user to make more precise energy density adjustments than an apparatus generating only focused shock waves, which is generally limited in terms of lowering the energy output. Nevertheless, in some cases the first use of a high energy focused shock wave targeting a treatment zone may be the best approach followed by a transmission of lower energy unfocused wave patterns.

In the use of reflexology zones as the indirect pathway or gate to stop infection and cure diseases/disorders and/or control pain response, the present invention has actual empirical data showing the effectiveness in the zone directed to a bone. It is therefore further believed that similar modulation and beneficial adjustment can be achieved at other reflexology zones for stimulating, modulating or adjusting other glands, bones, nerves or organs such as the liver, kidney or any of those indicated in FIG. 13 for the foot zones and FIG. 14 for the hand zones. It is further believed that the hybrid Eastern medical acupuncture treatments or massages historically used are far less effective and less reliable than the results achieved by the deeper tissue penetrating transmission that are achieved by acoustic shock wave therapy applied to these reflexology zones.

Included in treatments for infections are all auto immune indications/disorders as well as disorders of chronic local and systemic inflammation, congestive heart or lung failure. Mechanism is reduction of any systemic inflammation, drastically lower the white blood cell count and causing the body to stop attacking itself. Treatments can be applied weekly to hands and feet, maximum 2500 each, treating the entire foot or hand, focusing on those painful zones until the pain disappears or decreases substantially, preferably treating for 4 weeks or less. Also, if patient has heart inflammation/congestive heart failure, focusing on the known reflexology zones for hearts and lungs. These spots may be painful at first.

When treating the hands and feet and noting the painful spots, one can locate areas on the known reflexology zone charts to diagnose weaknesses, or injuries in the body at each corresponding zone.

Reflexology methods of treating infections using both feet and hands to generate total wellness, and more specifically this treatment reduces inflammation systemically. No device can do that. This reduction in systemic inflammation cures all auto immune disorders. A body stops attacking itself. Reflexology treats a specific part of the foot to treat a specific infected target/organ. Treating all of the zones resets a body’s overall wellness.

In the application of reflexology treatment of the present invention, pathologic tissue can be targeted directly and in combination with the named identifiable reflexology zones which is believed to be the best possible therapy. Shock wave or pressure pulse treatment can cure any part of the body by treating using the combination of directly treating the tissue and also treating the appropriate reflexology zone. One can also diagnose bodily deficiencies/injuries/pathologies by pulsing all of the reflexology zones of the hands and feet and noting the painful areas. These painful areas will correspond to a pathologic location in the body. The heart zone will hurt in a congestive heart failure patient. Continue to treat this painful spot until inflammation is gone and the appropriate biologic cascade has been activated in the heart.

There are two main groups of adrenoreceptors, α and β, with 9 subtypes in total: α are divided to α1 (a Gq coupled receptor) and α2 (a Gi coupled receptor); α1 has 3 subtypes: α1A, alB and α1D; α2 has 3 subtypes: α2A, α2B and α2C; β are divided to β1, β2 and β3. All 3 are coupled to Gs proteins, but β2 and β3 also couple to Gi. Gi and Gs are linked to adenylyl cyclase. Agonist binding thus causes a rise in the intracellular concentration of the second messenger cAMP. Gi inhibits the production of cAMP. Downstream effectors of cAMP include cAMP-dependent protein kinase (PKA), which mediates some of the intracellular events following hormone binding. Epinephrine (adrenaline) reacts with both α- and β-adrenoreceptors, causing vasoconstriction and vasodilation, respectively. Although α receptors are less sensitive to epinephrine, when activated at pharmacologic doses, they override the vasodilation mediated by β-adrenoreceptors because there are more peripheral α1 receptors than β-adrenoreceptors. The result is that high levels of circulating epinephrine cause vasoconstriction. However, the opposite is true in the coronary arteries, where β2 response is greater than that of α1, resulting in overall dilation with increased sympathetic stimulation. At lower levels of circulating epinephrine (physiologic epinephrine secretion), β-adrenoreceptor stimulation dominates since epinephrine has a higher affinity for the β2 adrenoreceptor than the α1 adrenoreceptor, producing vasodilation followed by decrease of peripheral vascular resistance. Smooth muscle behavior is variable depending on anatomical location. One important note is the differential effects of increased cAMP in smooth muscle compared to cardiac muscle. Increased cAMP will promote relaxation in smooth muscle, while promoting increased contractility and pulse rate in cardiac muscle.

α receptors have actions in common, but also individual effects. Common or still receptor unspecified actions include: vasoconstriction and decreased motility of smooth muscle in gastrointestinal tract. Subtype unspecific α agonists can be used to treat rhinitis, they decrease mucus secretion. Subtype unspecific α antagonists can be used to treat pheochromocytoma, they decrease vasoconstriction caused by norepinephrine.

α1-adrenoreceptors are members of the Gq protein-coupled receptor superfamily. Upon activation, a heterotrimeric G protein, Gq, activates phospholipase C (PLC). The PLC cleaves phosphatidylinositol 4,5-bisphosphate (PIP2), which in turn causes an increase in inositol triphosphate (IP3) and diacylglycerol (DAG). The former interacts with calcium channels of endoplasmic and sarcoplasmic reticulum, thus changing the calcium content in a cell. This triggers all other effects, including a prominent slow after depolarizing current (sADP) in neurons. Actions of the α1 receptor mainly involve smooth muscle contraction. It causes vasoconstriction in many blood vessels, including those of the skin, gastrointestinal system, kidney, renal artery, and brain. Other areas of smooth muscle contraction are: ureter, vas deferens, hair (arrector pili muscles), uterus (when pregnant), urethral sphincter, urothelium and lamina propria, bronchioles (although minor relative to the relaxing effect of β2 receptor on bronchioles), blood vessels of ciliary body (stimulation causes mydriasis). Actions also include glycogenolysis and gluconeogenesis from adipose tissue and liver; secretion from sweat glands and Na+ reabsorption from kidney, α1 antagonists can be used to treat: hypertension, they decrease blood pressure by decreasing peripheral vasoconstriction and benign prostate hyperplasia, they relax smooth muscles within the prostate thus easing urination.

The α2 receptor couples to the Gi/o protein. It is a presynaptic receptor, causing negative feedback on, for example, norepinephrine (NE). When NE is released into the synapse, it feeds back on the α2 receptor, causing less NE release from the presynaptic neuron. This decreases the effect of NE. There are also α2 receptors on the nerve terminal membrane of the post-synaptic adrenergic neuron. Actions of the α2 receptor include: decreased insulin release from the pancreas, increased glucagon release from the pancreas, contraction of sphincters of the GI-tract, negative feedback in the neuronal synapses - presynaptic inhibition of norepinephrine release in CNS, increased platelet aggregation (increased blood clotting tendency), decreases peripheral vascular resistance. α2 agonists can be used to treat: hypertension, they decrease blood pressure raising actions of the sympathetic nervous system, impotence, they relax penile smooth muscles and ease blood flow and depression, they enhance mood by increasing norepinephrine secretion.

Subtype unspecific β agonists can be used to treat: heart failure, they increase cardiac output acutely in an emergency, circulatory shock, they increase cardiac output thus redistributing blood volume, and anaphylaxis - bronchodilation. Subtype unspecific β antagonists, beta blockers, can be used to treat: heart arrhythmia, they decrease the output of sinus node thus stabilizing heart function, coronary artery disease, they reduce heart rate and hence increasing oxygen supply, heart failure, they prevent sudden death related to this condition which is often caused by ischemias or arrhythmias, hyperthyroidism, they reduce peripheral sympathetic hyperresponsiveness, migraine, they reduce number of attacks, stage fright, they reduce tachycardia and tremor, glaucoma, they reduce intraocular pressure.

Actions of the β1 receptor include: increase cardiac output by increasing heart rate (positive chronotropic effect), conduction velocity (positive dromotropic effect), stroke volume (by enhancing contractility - positive inotropic effect), and rate of relaxation of the myocardium, by increasing calcium ion sequestration rate (positive lusitropic effect), which aids in increasing heart rate; increase renin secretion from juxtaglomerular cells of the kidney and increase ghrelin secretion from the stomach.

β2 adrenoreceptor (PDB: 2rh1) stimulates cells to increase energy production and utilization. Actions of the β2 receptor include: smooth muscle relaxation throughout many areas the body, e.g. in bronchi (bronchodilation, see salbutamol), GI tract (decreased motility), veins (vasodilation of blood vessels), especially those to skeletal muscle (although this vasodilator effect of norepinephrine is relatively minor and overwhelmed by α adrenoceptor-mediated vasoconstriction), lipolysis in adipose tissue, anabolism in skeletal muscle, relax non-pregnant uterus, relax detrusor urinae muscle of bladder wall, dilate arteries to skeletal muscle, glycogenolysis and gluconeogenesis, stimulates insulin secretion, contract sphincters of GI tract, thickened secretions from salivary glands, inhibit histamine-release from mast cells, increase renin secretion from kidney, and involved in brain - immune communication. β2 agonists can be used to treat: asthma and COPD, they reduce bronchial smooth muscle contraction thus dilating the bronchus, hyperkalemia, they increase cellular potassium intake, and preterm birth, they reduce uterine smooth muscle contractions.

Actions of the β3 receptor include: increase of lipolysis in adipose tissue. β3 agonists could theoretically be used as weight-loss drugs, but are limited by the side effect of tremors.

Shock wave or pressure pulse treatment can modulate alpha 1 and 2, beta, and other adrenergic receptors by directly targeting the tissue AND by the stimulation of the reflexology zones. For example, by targeting the hearts reflexology zones you can modulate alpha receptors in the heart. Shock wave or pressure pulse treatment can recruit, activate and differentiate stem cells by directly targeting the pathologic tissue or by targeting the pertinent reflexology zones or preferably by doing both in combination. This is the same for modulating inflammation locally by the direct targeting or modulating SYSTEMIC inflammation by treating any or all of the reflexology zones.

Inflammation equals disease and can cause cancer. Chronic inflammation will develop into cancer, especially in the prostate. All shock waves or pressure pulses collectively referred to as acoustic waves, including radial, focused or unfocused, in the desired energy levels in the targeted or pathologic tissues, can reduce inflammation in tissues, organs, pre skin cancers, and prevent or delay the onset of cancer. Preventative re-treatments every 90 days or at least annually is recommended. Treating the infected heart or other tissues can prevent or delay heart or other disease related issues. In the case of arterial vascular plaque buildup, the treatment by sound waves allows the plaque to either dislodge or be absorbed by the stimulated cells allowing the body to naturally flush the plaque out of the arteries.

It will be appreciated that the apparatuses and processes of the present invention can have a variety of embodiments, only a few of which are disclosed herein. It will be apparent to the artisan that other embodiments exist and do not depart from the spirit of the invention. Thus, the described embodiments are illustrative and should not be construed as restrictive.

Variations in the present invention are possible in light of the description of it provided herein. While certain representative embodiments and details have been shown for the purpose of illustrating the subject invention, it will be apparent to those skilled in this art that various changes and modifications can be made therein without departing from the scope of the subject invention. It is, therefore, to be understood that changes can be made in the particular embodiments described which will be within the full intended scope of the invention as defined by the following appended claims.

What is claimed is:

1. A method of treating a patient having cancer cells comprising the step of:
directing sound energy from one or more acoustic shock wave or pressure pulse treatments into the patient targeting the cancer cells in at least one tumor to cause the patient’s immune system to identify the cancer cells as foreign objects and trigger a natural healing mechanism of the patient’s immune system to destroy the foreign objects, and
wherein targeting the at least one tumor initiates cellular communication resulting in an unmasking of all cancer cells in the patient’s body by destroying a cloaking capability of the cancer cells which made the cancer cells indistinguishable from normal cells allowing the cancer cells to hide from the immune system prior to the one or more acoustic shock wave or pressure pulse treatments.

2. The method of claim 1, wherein treating the at least one tumor causes the cancer cells to secrete exosomes into extracellular spaces with MRNA that triggers a biologic cascade and cellular communication or signaling alerting the immune system to identify the cancer cells as foreign objects to target and destroy the cancer cells.

3. The method of claim 2, wherein the cellular communication or signaling is a secretion by the cancer cells to send exosomes outside of the cancer cells with proteins that the unmasking the cloaking capability of the cancer cells initiates causing the biologic cascade.

4. The method of claim 3, wherein during the acoustic shock wave or pressure pulse treatment no cancer cells are destroyed by the sound energy, however soon thereafter the unmasking the cancer cells in the at least one tumor allows the immune system to attack all cancer cells.

5. The method of claim 1 further comprises treating a reflexology zone to reduce systemic inflammation to accelerate cancer recovery.

6. The method of claim 1, wherein the treatment includes an improved long and short term blood supply, these mechanisms include both short and long term improvements in blood supply, an up regulation of anti-microbial peptides, especially peptide LL 37, a disruption of biofilms that protect these foreign objects, and an increase in cellular communication such that healthy cells identify these foreign objects as targets of the body’s natural defenses, as the improved blood supply allows a body to deliver natural defenses and increases the supply of medications administered by a physician or other medications, a disruption of cellular membranes, increased cell membrane permeability, and an improvement in cellular communication causing the patient’s immune system to identify and attack the cancer cells and further causes recruiting or stimulating an increase in anti-microbial peptides.

7. The method of claim 1 further comprises the step of:
administering one or more of medications, chemotherapy, radiation or checkpoint inhibitors to the patient including, but not limited to anti-viral medications, antibiotics, anti-fungal medications or anti-mold medications or anti-cancer medications, wherein the sound wave treatment improves the utility of these medications by increasing the amounts of these medications to the affected cells by increasing the short term and permanent blood supply to the cells and increasing the cellular communication to cause the body to aid in the fight against the foreign material.

8. The method of claim 3, wherein the treatment increases the permeability of the patient’s cell membranes allowing an increase in releasing anti-microbial peptides and inflow of the medications into the cells while increasing the blood supply toward the infection.

9. The method of claim 3, wherein the treatment is provided prior to, during or after administering medications, chemotherapy, radiation or checkpoint inhibitors or any combination thereof.

10. The method of claim 9, wherein the dosages or strength of the medications can be reduced when used in combination with the treatment.

11. The method of claim 1, wherein the acoustic shock waves or pressure pulses are focused or non-focused, convergent, divergent, planar or nearly planar, radial or spherical, shaped, linear or otherwise reflected or directed and are emitted by a generator or any mechanical device, wherein the generator or mechanical device is one of a radial, a spherical, a ballistic, a linear, a piezoelectric, or an electrohydraulic or electromagnetic generator.

12. The method of claim 1 wherein the treatment can be administered with or without cavitation.

13. A method of treating a patient having cancer cells comprising the step of:
directing sound energy from one or more acoustic shock wave or pressure pulse treatments into the patient targeting the cancer cells in the patient’s blood to cause the patient’s immune system to identify the cancer cells as foreign objects and trigger a natural healing mechanism of the patient’s immune system to destroy the foreign objects, and
wherein targeting the blood initiates cellular communication resulting in an unmasking of all cancer cells in the patient’s body by destroying a cloaking capability of the cancer cells which made the cancer cells indistinguishable from normal cells allowing the cancer cells to hide from the immune system prior to the one or more acoustic shock wave or pressure pulse treatments.

14. The method of claim 13, wherein treating the blood causes the cancer cells to secrete exosomes into extracellular spaces with MRNA that triggers a biologic cascade and cellular communication or signaling alerting the immune system to identify the cancer cells as foreign objects to target and destroy the cancer cells.

15. The method of claim 14, wherein the cellular communication or signaling is a secretion by the cancer cells to send exosomes outside of the cancer cells with proteins that the unmasking the cloaking capability of the cancer cells initiates causing the biologic cascade.

16. The method of claim 15, wherein during the acoustic shock wave or pressure pulse treatment no cancer cells are destroyed by the sound energy, however soon thereafter the unmasking the cancer cells allows the immune system to attack all cancer cells.

17. The method of claim 13 further comprises treating a reflexology zone to reduce systemic inflammation to accelerate cancer recovery.

18. The method of claim 13, wherein the treatment includes an improved long and short term blood supply, these mechanisms include both short and long term improvements in blood supply, an up regulation of anti-microbial peptides, especially peptide LL 37, a disruption of biofilms that protect these foreign objects, and an increase in cellular communication such that healthy cells identify these foreign objects as targets of the body’s natural defenses, as the improved blood supply allows a body to deliver natural defenses and increases the supply of medications administered by a physician or other medications, a disruption of cellular membranes, increased cell membrane permeability, and an improvement in cellular communication causing the patient’s immune system to identify and attack the cancer cells and further causes recruiting or stimulating an increase in anti-microbial peptides.

19. The method of claim 13 further comprises the step of:
administering one or more of medications, chemotherapy, radiation or checkpoint inhibitors to the patient including, but not limited to anti-viral medications, antibiotics, anti-fungal medications or anti-mold medications or anti-cancer medications, wherein the sound wave treatment improves the utility of these medications by increasing the amounts of these medications to the affected cells by increasing the short term and permanent blood supply to the cells and increasing the cellular communication to cause the body to aid in the fight against the foreign material.

20. The method of claim 16, wherein the treatment increases the permeability of the patient’s cell membranes allowing an increase in releasing anti-microbial peptides and inflow of the medications into the cells while increasing the blood supply toward the infection.

21. The method of claim 16, wherein the treatment is provided prior to, during or after administering medications, chemotherapy, radiation or checkpoint inhibitors or any combination thereof.

22. The method of claim 19, wherein the dosages or strength of the medications can be reduced when used in combination with the treatment.

23. The method of claim 13, wherein the acoustic shock waves or pressure pulses are focused or non-focused, convergent, divergent, planar or nearly planar, radial or spherical, shaped, linear or otherwise reflected or directed and are emitted by a generator or any mechanical device, wherein the generator or mechanical device is one of a radial, a spherical, a ballistic, a linear, a piezoelectric, or an electrohydraulic or electromagnetic generator.

24. The method of claim 13, wherein the treatment can be administered with or without cavitation.

25. The method of claim 13, wherein treating the blood to initiate an immune response by targeting the heart where blood pools before pumping or by targeting the large vessels in the groin or even targeting the patient’s hands and/or feet to initiate the immune response to destroy circulating cancer and virus cells in the blood, outside of a tumor and get the same response as if targeting the tumor.

26. The method of claim 13 further comprises the steps of:
drawing blood into an IV bag and clamping it off from the patient;
treating the blood with 1000 pulses thoroughly treating the blood with the acoustic shock waves or pressure pulses in a volume that cannot harm the patient when removed; and
after treating the blood, returning the treated blood to the patient with another IV line and drawing a next portion until most of the blood in the patient has been treated, wherein the sound energy is in the range of 0.001 mJ/mm² and 0.30 mJ/mm².
